(19) United States
(12) Patent Application Publication
(10) Pub. No.: US 2014/0023728 A1
(43) Pub. Date: Jan. 23, 2014

(54) Iodine Liquid Soap, Dispenser and Method

(71) Applicant: David Ricketts, SR., Irvine, CA (US)

(72) Inventor: David Ricketts, SR., Irvine, CA (US)

(21) Appl. No.: 13/554,875

(22) Filed: Jul. 20, 2012

(51) Int. Cl. A61K 33/18; A47K 5/14

(52) U.S. Cl. CPC A61K 33/18; A47K 5/14
USPC 424/667; 222/190

(57) ABSTRACT

A iodine liquid soap dispenser having a reservoir containing an iodine solution. The dispenser has a head communicating with the reservoir capable of delivering the solution to the exterior of the reservoir in the form of foam. The solution comprises water, iodine and iodide, and contains iodine in a range of concentration between 0.35% and 0.05% by weight of the solution. The solution may also contain one or more of the following: an anionic surfactant, Hydrogen Iodide, glycerine, and sodium hydroxide.

### FIELD OF THE INVENTION

This invention relates generally to liquid soaps for hand and face. In particular, the invention relates to an iodine-based liquid hand and face soap, the liquid soap in a foaming dispenser, and the method of applying the soap.

### BACKGROUND OF THE INVENTION

Iodine in solution is known as an antiseptic and disinfectant, and as an emergency disinfectant of drinking water. Elemental iodine in molecular form is nonpolar. As a result, it is poorly soluble in water, with one gram dissolving in 3450 ml at 20 degrees Celsius and 1280 ml at 50 degrees Celsius. However, the solubility of elemental iodine in water soluble is greatly increased by adding iodide to produce triiodide anion (I₃⁻). In an aqueous solution, the elemental iodine molecules exist in an equilibrium state with the triiodide ions. An example of such a solution is Lugol's iodine, also known as Lugol's solution. It consists of elemental iodine and potassium iodide in water.

Within the last few decades, variations of Lugol's solution have been used in veterinary applications. For example, my U.S. Pat. No. 5,534,266 discloses an aqueous iodine composition for topical application to dairy cow teats. Treatment with such a composition is disclosed as reducing the spread of mastitis infection, improving teat appearance and skin condition, and promoting the “milk let down” reflex and the release of milk. Typically, teat dips use solutions containing about 1% iodine.

More recently, it was contemplated that teat dips would be applied by an apparatus that produces a foam. U.S. Pat. No. 6,935,271, issued to Dennis Edison, et al., a method for producing a foamed bovine teat dip by providing a teat cup to surround the teat and providing to the cup the dip in the form of a foam. According to the patent, the foam produced by the disclosed apparatus adheres to the teat and udder area without significant run off and forms a bead at the end of the teat. Col. 3, line 45 et seq. The use of foam reduces runoff, affording a longer period of contact and protection against bacterial infection.

Another veterinary application of iodine has been recognized for wound healing. Iodine “wound sprays” are commercially available as non-stinging antiseptics for non-human animals. A 1% titratable iodine is available from Priority Care, for this purpose. A 2.44% minimum titratable iodine is available from Centaur, Inc.

An example of a human use of iodine is CELLUMEND, a product sold to treat cellulite. It purportedly includes “high amounts” of iodine from herbal ingredients such as Fucus, Laminaria and Carragaheen. The product also includes Hydriodidic Acid, which promotional literature claims is a skin conditioning agent and provides effective lipolytic properties, “since it stimulates lipases.”

According to its label, the CELLUMEND product relies on expensive sources of iodine found in relatively small quantities in herbs. Iodine used in medicinal and industrial applications, in comparison, is typically obtained from iodine-containing brines obtained in natural gas production, and is significantly less expensive.

As mentioned above, concentrations of iodine at 1% or higher are recognized as being effective for veterinary application. However, prolonged, direct human skin contact with iodine, at such high concentrations, as might occur in cleaning and antiseptic applications, risks thyroid damage. Moreover, periodic contact to iodine at such concentrations causes some people to develop sensitivity to iodine, a rash, or even burns.

Conventional wisdom prior to the current innovation is that only one of the iodine-related constituents in a Lugol's-type solution provides the various medical and cosmetic benefits. To obtain the benefits, the concentration of that constituent must be sufficiently high at the point and time of application. However, when applying a solution at those concentrations, the concentrations of other iodine-related constituents are also raised. Disadvantages, such as tissue damage, results from the presence of other iodine constituents at those relatively high concentrations.

### SUMMARY OF THE INVENTION

I have developed a method for using a relatively inexpensive, iodine solution as a soap for periodic human use that obtains the benefits of the type recognized in veterinary uses, but reduces the risks to humans. My method accomplishes this by increasing the relative concentration of the triiodide (I₃⁻) constituent at the point of contact with the human skin. This allows for the use of non-herbal iodine at a substantially lower concentration than previously thought would provide therapeutic benefits. The method utilizes foaming of the liquid. It is believed that the surface tension of the bubbles in the foam creates an micro-environment that significantly increases the relative concentration of the triiodide (I₃⁻) constituents. The concentration of iodine in the pre-foamed liquid is greatly reduced, and the risk of tissue damage or other disadvantages of periodic human skin contact is reduced.

My invention also contemplates a liquid soap dispenser for the application of the iodine-based liquid soap as foam. The dispenser has a reservoir that holds a dilute iodide-iodine solution. The device has a dispensing head that communicates with the reservoir and is capable of simultaneously foaming the liquid soap and delivering it exterior to the device. Use of the dispenser provides the benefits of an iodine-based liquid soap without skin exposure to the high concentrations of constituents that pose the risk of tissue damage.

### BRIEF DESCRIPTION OF THE DRAWINGS

FIG. 1 is a perspective view of the liquid soap dispenser containing the iodine liquid soap and contemplated for an embodiment of the invention; and,

FIG. 2 is a perspective view of an embodiment of the invention, shown in FIG. 1, with clear, protective cap removed and foaming dispenser head depressed to dispense the liquid soap as a foam.

### DETAILED DESCRIPTION OF THE PREFERRED EMBODIMENTS

According to the invention, an effective iodine-containing liquid soap for human use is accomplished with dilute iodine by applying the solution as foam. The foam increases the effectiveness of the iodine, allowing the amount of iodine in the soap solution to be significantly reduced. The preferred embodiment dilutes the iodine by a factor of about 6 as compared to traditional iodine solutions used to treat the skin of non-human animals, such as a teat dip.

The preferred embodiment of the solution contains several chemical components. The solution is an aqueous solution of iodine. Iodide, preferably in the form of Hydriodic Acid, an aqueous solution of Hydrogen Iodide or HI, is provided to increase the solubility of iodine in water, consistent with the teaching of Lugol. Alternatively, other sources of iodide, such as potassium iodide, can be used as a substitute for some or all of the Hydrogen Iodide used in the composition.

The solubility of iodine is maximized by including iodide with iodine in a weight ratio in the range of about 1:1 to about 2:1. In other words, the solution should have approximately the same amount or more of iodide as iodine by weight, but not much more than twice as much. The presence of iodide with iodine in water forms triiodide (I₃⁻). It is believed that the triiodide (I₃⁻) ion provides some or all of the health benefits associated with contact with veterinary use of 1% iodine. Periodic washing with triiodide provides skin with a healthy, resilient feel and a youthful appearance.

Other constituents formed from an iodine-iodide aqueous solution are believed to provide the disadvantages of iodine exposure mentioned above. Such risks are concentration dependent. Reducing the concentration of iodine-iodide constituents in the solution reduces the risks.

The benefits of exposing the skin to periodic triiodide (I₃⁻) treatments is obtained by applying triiodide (I₃⁻) in solution in the form of foam. Transforming the solution to foam before applying it increases the effectiveness of the triiodide ion (I₃⁻), possibly because of the effect of surface tension at the surface of foam bubbles. Hence, the amount of triiodide (I₃⁻) at the point of application to the skin can be similar to that experienced by a 1% iodine wash, with about ⅓^{rd} to about 1/20^{th} the amount of iodine in solution. This significantly reduces the amount of other, undesirable constituents in an aqueous iodine-iodide solution, and provides an iodine-containing liquid soap product that is acceptable for periodic human skin contact. It also significantly reduces the expense of manufacture of the iodine-containing liquid soap.

The preferred embodiment of the solution may be improved by adjusting the pH to a range of between about 2.5 and about 4.5, and preferably about 3.5. This pH range maximizes the solubility of iodine in the presence of iodide. Again, this allows for the desired presence of sufficient triiodide (I₃⁻) in solution at the point of contact with skin, while using a relatively dilute solution of iodine that is acceptable for periodic human skin contact. It also provides for a more acceptable and effective hand and face wash.

The desired pH is accomplished by the addition of an alkaline, buffering agent, preferably sodium hydroxide (NaOH), also known as lye and caustic soda. It is a caustic metallic base that is soluble in water. Alternatively, other alkaline buffers known to those skilled in the art may be used to achieve the desired pH. The presence of Citric Acid will also impact the pH of the soap.

The preferred embodiment of the solution may also contain one or more anionic surfactants. The surfactants act as emulsifiers/dispersants appropriate for use in personal care products to improve skin feel. Such surfactants also may assist in the transformation of the solution to foam, and the cleaning of the skin surface, i.e., removal of dirt and grime. Typically, the solution will contain anionic surfactants in the weight percentage range of about 3% to about 10%.

These surfactants include one or more polyoxyethylene alkylether carboxylic acids. Such anionic surfactants are commercially available from Kao Chemicals Europe as part of its AKYPO® RLM series of products. Preferrable polyoxyethylene alkylether carboxylic acids include one or both of Laureth-11 Carboxylic Acid and Laureth-6 Carboxylic Acid. Laureth-11 Carboxylic Acid is commercially available as Kao Chemical's AKYPO® RLM 100 product. Laureth-6 Carboxylic Acid is commercially available as Kao Chemical's tradename AKYPO® RLM 45 CA.

Another anionic surfactant that may be used in the solution contemplated for use in the invention is sodium lauryl sulfate (also know as Carsonol SLS, sodium dodecyl sulfate (SDS or NaDS) or sodium laurilsulfate). It is an organosulfate having a 12-carbon tail attached to the sulfate group, giving the material amphiphilic properties required of a detergent. It can be used in addition to other anionic surfactants such as the polyoxyethylene alkylether carboxylic acids described above. When using polyoxyethylene alkylether carboxylic acids in combination with sodium lauryl sulfate, the total amount of anionic surfactants is preferably in the weight percent range of about 5% to about 8%.

The contemplated inventive liquid soap may also contain glycerin. Glycerin is a humectant and attracts moisture to skin. It is a natural by-product making. However, commercial soap manufacturers remove glycerin from soap for use in more profitable products, such as lotions and creams. Its presence in my contemplated invention increases the benefits of the liquid soap products contemplated herein. Typically, the contemplated iodine solution contains less than 2% glycerin, and preferably about one-half percent.

As mentioned above, administering the iodine-containing solution as foam allows the benefits generally associated with much higher concentrations of iodine in solution to be achieved with substantially lower iodine concentrations. The benefits of my contemplated invention are obtainable at iodine concentrations as high as about 0.35% and as low as about 0.05%. Concentrations below about 0.05% will begin to lose effectiveness even after being transformed to foam. Preferably, the range would be about 0.25% to about 0.10%. More preferably, the range would be about 0.20% to about 0.14%.

A preferred solution would contain the combination of the following ingredients:

Water

Laureth-11 Carboxylic Acid

Sodium Lauryl Sulfate (Carsonol SLS)

Laureth-6 Carboxylic Acid

Glycerin

Citric Acid

Sodium Hydroxide

Hydrogen Iodide (HI)

Iodine

Alternatively, one or more of the surfactants could be substituted with other types of surfactants or be deleted from the solution. The use of glycerin also is optional.

The solution described above can be applied to human skin as foam by use of a commercially available product that transforms liquid into foam. Such a dispenser is the PureFoam™ non-aerosol foamer, 43 mm size, that delivers 0.70 ml of output per single pump action.

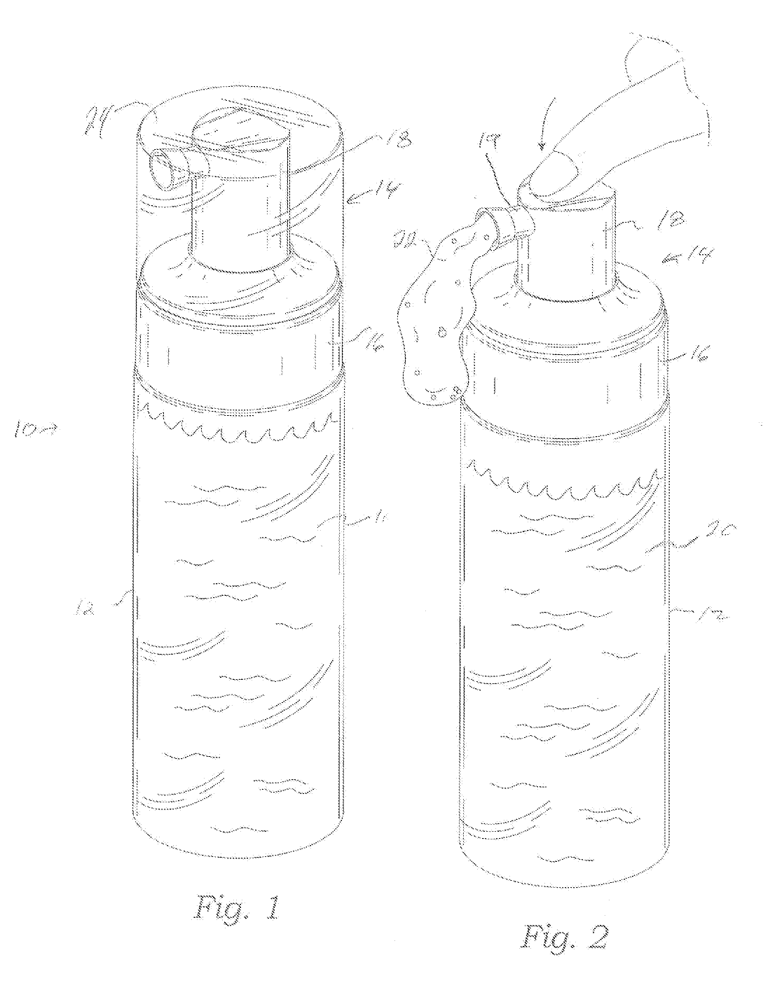

As shown in FIGS. 1 and 2, a dispenser 10, includes a reservoir 12. The reservoir 12 is generally cylindrical with a closed, flat bottom and an open top containing a lip with screw threads. The dispenser also has a head 14 that has collar portion 16 and pump portion 18. The collar portion 16 has an open bottom also containing a lip with screw threads. The top of reservoir 12 is capable of being connected to the bottom of collar portion 16 of head 14 by mating the two sets of threaded lips. When connected by being screwed together the contents of the reservoir 12 securely communicate with the head 14 without leakage.

The pump portion 18 of the head 14 is moveable relative to the collar portion 16, and contains a nozzle 19. The pump portion 18 communicates with the interior of and is downwardly displaceable relative to the collar portion 16. As shown in FIG. 2, such displacement delivers the liquid in the reservoir through the head 14 and out of the nozzle 19 to the exterior of the dispenser 10. This action not only moves the liquid in the reservoir to the exterior of the dispenser, but also transforms it into foam. It is at this point, and in this form (i.e. foam), that the liquid soap is available for application to human skin.

As shown in FIG. 1, when not in use, the dispenser contains a cap 24. The cap 24 is cylindrical in cross-sectional shape with closed top and open bottom and is made of a clear plastic material to reveal the pump portion 18 of the head 14. The cap has a diameter similar to the diameter of the reservoir 12 and snugly mates with the collar portion 16 of the head 14 to obtain a pressure fit.

When present on the dispenser 10, the cap has at least three functions. First, it prevents unintended displacement and discharge of foam from the reservoir. Second, it may create an air-tight environment around the nozzle 19 and pump portion 18 of the head 14 that is otherwise exposed to the ambient environment. This reduces the loss of solution 20 from the reservoir 12 through head 14 due to evaporation over time. Third, it reduces potential damage to the head upon unintended impact.

As contemplated in the preferred embodiment of the current invention, the reservoir 12 holds an iodine-containing liquid soap solution 20 as described above and in the example below. When the pump portion 18 is depressed, the pump-action delivers the liquid soap 20 in the form of triiodide-containing foam 22. This foam 22 is available to be applied to the user's hands, face or skin of other body part. Although containing a relatively low concentration of iodine in the solution, when transformed into foam the relative benefits of the triiodide (I₃⁻) are increased beyond those obtained by application of the solution directly, i.e., without being transformed into foam.

### Example

A preferred way to employ the invention is to use a solution having the following composition:

| | |
|---|---|
| Water | 92.3% |
| Laureth-11 Carboxylic Acid | 3.7% |
| Sodium Lauryl Sulfate | 1.8% |
| Laureth-6 Carboxylic Acid | 0.9% |
| Glycerin | 0.4% |
| Citric Acid | 0.05% |
| Sodium Hydroxide | 0.3% |
| Hydrogen Iodide | 0.35% |
| Iodine | 0.17% |

The solution is put into the reservoir 12 of the dispenser 10 described above. The pump portion 18 of head 14 is depressed relative to collar portion 16 (for example, by a finger, shown in FIG. 2) to deliver solution as foam 22 to the exterior of dispenser 10. The foam 22 is then administered to the hand, face or other body part of a user.

Alternatively, the liquid soap may be delivered in the form of a shampoo. Foaming agents of the type conventionally used in shampoos can be added to the liquid solution and the foaming accomplished during administration. Such a liquid soap would be placed into the human skin or scalp and massaged to create suds. Some or all of the advantages of the invention described herein would be obtained.

It will be appreciated that the iodine solution, dispenser and method can be practiced in a variety of ways not limited to the preferred embodiments and examples described above without departing from the scope of my invention contemplated herein.

What is claimed is:

1. A liquid soap dispenser comprising:
a reservoir containing a solution;
a head communicating with the reservoir, said head capable of delivering the reservoir solution to the exterior of the reservoir as a foam;
wherein said solution comprises water, iodine and iodide; and,
wherein the solution contains iodine in a range of concentration between about 0.35% and 0.05% by weight of the solution.

2. The liquid soap dispenser of claim 1 wherein the concentration range of iodine is about 0.25% to about 0.10%.

3. The liquid soap dispenser of claim 1 wherein the concentration range of the iodine is about 0.20% to about 0.14%.

4. The liquid soap dispenser of claim 1 wherein the reservoir solution has a pH in the range between 2.5 and 4.5.

5. The liquid soap dispenser of claim 1 wherein the reservoir solution has a pH of about 3.5.

6. The liquid soap dispenser of claim 1 further comprising at least one anionic surfactant.

7. The liquid soap dispenser of claim 6 wherein the anionic surfactant is selected from the group consisting of laureth-6 carboxylic acid, laureth-11 carboxylic acid, sodium lauryl sulfate, and combinations thereof.

8. The liquid soap of claim 7 further comprising glycerin.

9. A liquid soap dispenser comprising:
a reservoir containing a solution;
a head communicating with the reservoir, said head capable of delivering the reservoir solution to the exterior of the reservoir as a foam;
wherein said solution comprises water, iodine, Hydrogen Iodide, glycerine, and a anionic surfactant selected from the group consisting of laureth-6 carboxylic acid, laureth-11 carboxylic acid, sodium lauryl sulfate, and combinations thereof; and,
wherein the iodine is less than 0.25% by weight of the solution and greater than 0.10%.

10. The liquid soap dispenser of claim 9 wherein the head has a pump portion and a collar portion.

11. The liquid soap dispenser of claim 9 wherein weight percentage of iodine is between about 0.20% and about 14% of the solution.

12. The liquid soap dispenser of claim 11 wherein the weight ratio of Hydrogen Iodide to iodine is between about 1:1 and about 2:1, and wherein the anionic surfactant is between about 5% and 8%.

13. The liquid soap dispenser of claim 9 further comprising a cap removably attachable to the head.

14. A method of applying an iodine-containing liquid soap to human skin, the liquid soap comprising water, iodide, iodine, an alkaline pH adjusting agent and an anionic surfactant, wherein the iodine comprised between 0.35% and 0.05% of the weight of the liquid soap, the method comprising the steps of foaming the liquid soap; and,
applying the foam to human skin.

15. The method of claim 14 wherein the alkaline pH adjusting agent comprises sodium hydroxide.

16. The method of claim 14 wherein the iodine concentration is in the range of between 0.25% and 0.10% by weight of the liquid soap.

17. The method of claim 16 wherein the anionic surfactant is selected from the group consisting of laureth-6 carboxylic acid, laureth-11 carboxylic acid, sodium lauryl sulfate, and combinations thereof

18. The method of claim 16 wherein the liquid soap further comprises glycerin and wherein the iodide is Hydrogen Iodide.

19. The method of claim 17 wherein the soap is about 0.35% Hydrogen Iodide and about 0.4% glycerin.

20. The method of claim 17 wherein the anionic surfactant comprised between about 5% and about 8% by weight of the liquid soap.
